(19) United States
(12) Patent Application Publication
(10) Pub. No.: US 2006/0135607 A1
(43) Pub. Date: Jun. 22, 2006

(54) Drug for improving prognosis for subarachnoid hemorrhage

(75) Inventors: Sei Kobayashi, Yamaguchi (JP); Michiyasu Suzuki, Yamaguchi (JP); Norio Ikeda, Yamaguchi (JP)

Correspondence Address: BIRCH STEWART KOLASCH & BIRCH, PO BOX 747, FALLS CHURCH, VA, 22040-0747, US

(73) Assignee: MOCHIDA PHARMACEUTICAL CO., LTD., Tokyo (JP)

(21) Appl. No.: 10/544,480

(22) PCT Filed: Feb. 2, 2004
(86) PCT No.: PCT/JP04/00989

§ 371 Date: Aug. 4, 2005

(30) Foreign Application Priority Data
Feb. 7, 2003 (JP) 2003-031144

(51) Int. Cl. A61K 31/202; A61K 31/22

(52) U.S. Cl. 514/546; 514/560

(57) ABSTRACT

It is intended to provide a drug for improving prognosis for subarachnoid hemorrhage which contains, as the active ingredient, at least one member selected from the group consisting of eicosapentaenoic acid (EPA) and pharmaceutically acceptable salts and esters thereof, and a method of improving prognosis for subarachnoid hemorrhage which comprises orally or gastrically administering the above drug.

### TECHNICAL FIELD

The present invention relates to a drug for improving prognosis of subarachnoid hemorrhage which contains as an active ingredient at least one member selected from the group consisting of an eicosapentaenoic acid (hereinafter, abbreviated as “EPA”) and pharmaceutically acceptable salts and esters thereof, and to a method of improving prognosis for subarachnoid hemorrhage using the above drug.

### BACKGROUND ART

Subarachnoid hemorrhage is due to the rupture of cerebral aneurysm and refers to the state that hemorrhage has occurred in the subarachnoid cavity. In this country, 12 out of 100,000 people a year have suffered subarachnoid hemorrhage. It is an extremely serious disorder, as has been reported that about 20 to 40% of them would die out.

In addition, as complications of subarachnoid hemorrhage, there are known rebleeding, cerebral vasospasm, hydrocephalus, and the like. As a clinical treatment for those complications, a surgical operation such as clipping or coiling is conducted for preventing rebleeding. In addition, a surgical operation such as ventricular drainage or VP shunt has been performed for the appearance of hydrocephalus.

The detailed occurrence mechanism of cerebral vasospasm has been less well understood and in the present circumstances there is no definitive therapeutic method. However, for preventing a spasm, an intraspinal injection of nicardipine hydrochloride (indwelling drainage in the cisterna) or the like is performed. Besides, when the spasm has occurred, a superselective intraarterial injection treatment with papaverine hydrochloride or the like is performed. As a drug for improving subarachnoid hemorrhage-postoperative cerebral vasospasm and corresponding cerebral ischemia symptom, an intravenous injection of Eril injection (generic name: fasudil hydrochloride) is used clinically.

On the other hand, in conjunction with cerebral vasospasm, EPA has been disclosed in publications. For example, in the literatures cited in JP-A 2001-261556, in which an inhibitor for an abnormal contraction of a smooth muscle, which contains EPA as an active ingredient, is disclosed, the above cerebral vasospasm is exemplified as one of the disorders to be caused by the abnormal contraction of the smooth muscle.

However, as far as the inventors of the present invention know, there is no report describing that EPA may enhance the action of improving the prognosis of subarachnoid hemorrhage, particularly its use in clinical application, and increasing a recovery rate as well as a survival rate in clinical application.

Subarachnoid hemorrhage is an extensively serious disorder of high lethality, so that such a disease may turn one into a vegetable state or often leave aftereffects in many cases even though one fortunately was not led to death. When subarachnoid hemorrhage was developed, as described above, a therapeutic agent for preventing or inhibiting cerebral vasospasm is mainly used as a treatment for the complication in addition to surgical operations for rebleeding and hydrocephalus. However, results of the therapeutic agents and therapeutic methods which are currently used in clinical application, particularly prognostic improvement effects, are insufficient. Therefore, the development of a drug having an effect of improving prognosis has been expected.

### DISCLOSURE OF THE INVENTION

Accordingly, an object of the present invention is to provide a drug that improves the prognosis of a patient with subarachnoid hemorrhage, which has been under the conditions described above, and enhances a recovery rate or a survival rate with respect to such a disease.

The inventors of the present invention have intensely studied a drug for improving the prognosis of a patient with subarachnoid hemorrhage and enhancing a recovery rate and a survival rate thereof. Finally, the inventors of the present invention have completed the present invention by finding out that the drug of the present invention containing EPA as an active ingredient has the above actions, in particular, an extremely higher lifesaving effect.

As described above, among the complications of subarachnoid hemorrhage, it is known that EPA has an inhibitory effect on cerebral vasospasm (on abnormal contraction of the smooth muscle). However, a cause to lead to death or critical state after subarachnoid hemorrhage is not always due to the cerebral vasospasm which is one of the complications thereof.

An aspect of the present invention relates to a drug for improving prognosis of subarachnoid hemorrhage (hereinafter, simply called a drug for improving prognosis), characterized by containing as an active ingredient at least one selected from the group consisting of eicosapentaenoic acid, pharmaceutically acceptable salts and esters thereof.

Further, another aspect of the present invention relates to a method of improving prognosis for subarachnoid hemorrhage using the drug for improving prognosis characterized by administering a drug containing as an active ingredient at least one selected from the group consisting of eicosapentaenoic acid, pharmaceutically acceptable salts and esters thereof, orally or through a stomach tube, from the time of before, during, and/or after an operation involved in subarachnoid hemorrhage.

Furthermore, another aspect of the present invention relates to a method of improving prognosis for subarachnoid hemorrhage using the drug for improving prognosis, particularly one characterized in that, from the time of before, during, and/or after the operation involved in subarachnoid hemorrhage, a drug containing as an active ingredient at least one selected from the group consisting of eicosapentaenoic acid and pharmaceutically acceptable salts and esters thereof and a cholagogue drug, particularly ursodeoxycholic acid, are administered through a stomach tube.

### BEST MODE FOR CARRYING OUT THE INVENTION

Hereinafter, the present invention will be described in detail. The term “subarachnoid hemorrhage” as used herein means a hemorrhage state in a subarachnoid cavity. The rupture of cerebral aneurysm occupies about 60-80% of the causes. In addition, but not particularly limited to, other causes include head injury, rupture of cerebral arteriovenous malformation, and moyamoya disease of children.

The term “prognosis improvement” as used herein means an improvement in disease state or lethality in comparison with the conventional therapy. For instance, as a specific example but not limited to, such an improvement can be found in a clinical parameter such as the Glasgow Outcome Scale (GOS) in comparison with the conventional therapy.

EPA to be used in the present invention may be one commercially available or any of those obtained by purifying fish oils, EPA-producing bacterial cells and the culture medium thereof by any of known methods such as a continuous distillation method, a urea adduct method, a liquid chromatography method, a supercritical fluid chromatography method, and a combination thereof. Besides, if necessary, the resulting EPA may be subjected to an esterification treatment to make it into an ester such as alkyl ester, including ethyl ester, or glyceride. In addition, it may be prepared in a salt form with an inorganic base such as a sodium salt or a potassium salt; an organic base such as a benzylamine salt or a diethylamine salt; or a basic amino acid such as an arginine salt or a lysine salt.

In the present invention, EPA includes the above salts and esters in addition to a free fatty acid unless otherwise specified. Where EPA is administered to a human or an animal, preferable is of pharmaceutically acceptable. Of those, EPA is preferably used in an ester form of ethyl eicosapentaenoate (hereinafter, abbreviated as EPA-E).

The drug for improving prognosis of the present invention may contain any of other fatty acids as an additional active ingredient together with EPA, not to mention that the pure product of EPA can be used for. Examples of those fatty acids include unsaturated fatty acids such as docosahexaenoic acid, docosapentaenoic acid, docosamonoenic acid, arachidonic acid, eicosatetraenoic acid, eicosatrienoic acid, eicosamonoenic acid octadecatetraenoic acid, α-linolenic acid, linoleic acid, oleic acid, palmitoleic acid, hexadecatetraenoic acid, hexadecatrienoic acid, and hexadecadienoic acid; or saturated acids such as behenic acid, arachidic acid, stearic acid, palmitic acid, and myristic acid. Furthermore, the exemplified fatty acids described above, in addition to the free forms thereof, each of them may be a salt with an inorganic base such as a sodium salt or a salt with an organic base such as a benzylamine salt, alternatively, an ester form of an alkyl ester such as ethyl ester or glyceride.

In the drug for improving prognosis of the present invention, when a fatty acid other than EPA is contained as an active ingredient, it is preferable to contain EPA with a content of 50% or more by weight, preferably 70% or more by weight, more preferably 85% or more by weight in the whole fatty acids. It is preferable that the content of arachidonic acid is small.

As a drug for improving prognosis of the present invention, the active ingredient (compound) may be administered without modification. In addition, the active ingredient may be administered after dissolving it in hot water or may be administered in a suspension through a stomach tube. It may be also prepared as a suitable pharmaceutical preparation in advance to administration. In pharmaceutical preparation, appropriate carriers or media and various additives, which are used generally, can be arbitrarily chosen and then combined so as to be used in combination. For instance, any of excipients, binders, lubricants, colorants, flavors, sterilized water, vegetable oils, harmless organic solvents, harmless solubilizers (e.g., glycerin and propylene glycol), emulsifiers, suspending agents (e.g., Tween 80 and a gum arabic solution), isotonizing agents, pH adjustors, stabilizers, soothing agents, and the like can be used if necessary.

In particular, as the EPA is highly unsaturated, the above pharmaceutical preparation preferably contains an antioxidant in an effective amount enough to prevent the oxidation of EPA. Examples of the antioxidant include butylated hydroxytoluene (BHT), butylated hydroxyanisole (BHA), propyl gallate, gallic acid, and pharmaceutically acceptable quinone and α-tocopherol.

Dosage forms of the pharmaceutical preparations include forms such as tablets, capsules, microcapsules, granules, fine granules, powders, liquid preparations for oral administration, suppositories, syrups, inhalants, eye drops, ointments, and injections (emulsive, suspended, and nonaqueous), or solid injections to be used in an emulsion or a suspension at the time of use.

According to the present invention, the active ingredient can be administered to a patient without modification or as a pharmaceutically prepared drug for improving prognosis without distinction of orally, through a stomach tube, intravenous or intraarterial, inhalation, instillation, intrarectal, intravaginal or external use. Of those, it is particularly preferable to administer orally by encapsulating in a capsule such as a soft capsule or a microcapsule. Alternatively, it may be intravenously or intraarterially administered in the form of an injection (emulsive, suspended, or nonaqueous) or a solid injection to be used in emulsion or suspension at the time of use.

Since EPA-E-containing soft capsule agent less express side effects and is commercially available as a safe therapeutic drug for arteriosclerosis obliterans and hyperlipemia, the high-purity EPA-E-containing soft capsule agent commercially available in Japan as Epadel and Epadel-S (both manufactured by Mochida pharmaceutical Co., Ltd.) may be used.

The drug for improving prognosis of the present invention can be administered at a dose sufficient to express an effect of interest and can be arbitrarily increased or decreased in consideration of the dosage form, administration method, the number of doses per day, the degree of symptoms, weight, age, and so on.

In a case of an oral administration, EPA is administered at a dose of 0.1 to 9 g per day, preferably 0.5 to 6 g per day, more preferably 1 to 3 g per day divided in three times. However, the total dose may be administered at once or in several times if necessary.

When the oral administration is difficult, the above dose of EPA can be dissolved or suspended in hot water and then administered through a stomach tube (Magen Sonde). EPA is preferably administered after a meal. However, in the case of difficulty in orally dietary intake under IVH control, for raising the absorption of EPA, a cholagogue drug such as ursodeoxycholic acid (trade name: Urso, manufactured by Mitsubishi Pharma Corporation, or the like) can be administered in combination. Specifically, for example but not limited to, there is a method in which 50 mg of Urso granule (manufactured by Mitsubishi Pharma Corporation) is administered through the stomach tube and after 15 minutes 600 mg of EPA previously dissolved or suspended in hot water is then administered through the stomach tube.

In a case of intravenous or intraarterial administration, EPA is administered at a dose of 1-200 mg, preferably 5-100 mg, further preferably 10-50 mg at once or in divided dosages. However, it can be continuously administered over hours to several days using a drip or infusion pump or the like if necessary.

For a dosage period of EPA and the dosage schedule thereof, it is possible to administer for an optional dosage period and an arbitrary dosage schedule from the time of before, during, and/or after the operation involved in subarachnoid hemorrhage to the time after discharge from a hospital through the length of hospitalization. Pharmaceutical preparations commercially available in the market at present are contraindicated in bleeding patients. Care should be exercised in preoperative and intraoperative administration. From point of view of avoiding the promotion of bleeding tendency, it is preferable to start the administration postoperatively. For a rough standard of the dosage period, it is preferable to continuously administer over 1 week, further preferably from 2 weeks to 1 year.

### EXAMPLES

Examples of the present invention will be described below. However, those examples do not restrict the present invention.

### Example 1

[Object and Method]

Sixty four patients with subarachnoid hemorrhage caused by the ruptured aneurysm and subjected to surgical operations by the third day after onset (22 males and 42 females of the ages from 30's-80's) were divided into two groups (each having 32 individuals) designated as an EPA-administration group and an EPA-non-administration group. In addition, no difference was recognized in their background factors of both groups.

Just after the operation, the EPA administration group received the administration of ethyl eicosapentaenoate (trade name: Epadel, manufactured by Mochida Pharmaceutical Co., Ltd.), 1,800 mg divided in three-doses a day for 14 days. Subsequently, the administration was continued during the hospital stay. At the stage of being impossible to dietary intake, tube-feedings were carried out. EPA suspended in hot water was administered through a stomach tube (Magen Sonde). At that time, for enhancing the absorption of EPA, 50 mg of Urso granule (manufactured by Mitsubishi Pharma Corporation) was administered through the stomach tube 15 minutes before EPA administration. At the stage of being possible to dietary intake, a commercial EPA preparation was administered orally. Furthermore, both groups were treated in combination with basic treatments on subarachnoid hemorrhage. However, some of the drugs were defined as prohibited substances.

[Results]

At the 30th day from the onset of subarachnoid hemorrhage, an evaluation was carried out by the Glasgow Outcome Scale (GOS). The results are listed in Table 1.

TABLE 1

| | GOS | | | | |
|---|---|---|---|---|---|
| | GR | MD | SD | VS | D |
| EPA-non-administration group | 13 | 4 | 6 | 1 | 8 |
| EPA-administration group | 21 | 6 | 2 | 2 | 1 |

GR: Good recovery
MD: Moderate disability
SD: Severe disability
VS: Vegetative state
D: Death

As shown in Table 1, the EPA-administration group showed good results. As a result of the Wilcoxon U test, the P value was P=0.0128, resulting in significant difference between both groups.

In addition, the survival rates are listed in Table 2.

TABLE 2

| | Survival rate | | |
|---|---|---|---|
| | | | Survival |
| | Death | Survival | rate |
| EPA-non-administration | 8 | 24 | 75% |
| group | | | |
| EPA-administration group | 1 | 31 | 97% |

As shown in Table 2, it was confirmed that an extremely high survival rate, i.e., an extremely high lifesaving effect, can be obtained in the EPA-administration group. As a result of the Fisher's exact test, the P value obtained was P=0.0265, and thus a significant difference was found between both groups.

The adverse event considered as a result of the EPA administration was not found as well at all.

From the above description, it was confirmed that EPA improves the prognosis in a subarachnoid hemorrhage patient and has an effect of increasing a recovery rate and a survival rate.

### INDUSTRIAL AVAILABILITY

A drug for improving prognosis of subarachnoid hemorrhage is characterized by that, as active ingredient, at least one selected from the group consisting of eicosapentaenoic acid and pharmaceutically acceptable salts and esters thereof improves the prognosis in a subarachnoid hemorrhage patient and has an effect of increasing a recovery rate and a survival rate.

1. A drug for improving prognosis of subarachnoid hemorrhage, containing as an active ingredient at least one member selected from the group consisting of eicosapentaenoic acid, pharmaceutically acceptable salts and esters thereof.

2. A method of improving prognosis for subarachnoid hemorrhage, comprising administering a drug containing as an active ingredient at least one member selected from the group consisting of eicosapentaenoic acid, pharmaceutically acceptable salts and esters thereof, orally or through a stomach tube, from a time of before, during, and/or after an operation involved in subarachnoid hemorrhage.
